(19) United States
(12) Patent Application Publication
(10) Pub. No.: US 2019/0358081 A1
(43) Pub. Date: Nov. 28, 2019

(54) Cryo-Gastrointestinal Activator

(71) Applicant: Carl Frederick Dietrich, III, Chewelah, WA (US)

(72) Inventor: Carl Frederick Dietrich, III, Chewelah, WA (US)

(21) Appl. No.: 15/989,145

(22) Filed: May 24, 2018

(51) Int. Cl. A61F 7/10; A61F 7/02

(52) U.S. Cl. CPC A61F 7/10; A61F 2007/0028; A61F 7/02

(57) ABSTRACT

Specifically, the invention relates to the discovery and development of an effective cryogenic gastrointestinal triggering device that artificially activates the gastrointestinal tract's involuntary intestinal functions in the application process. Generally, several of the invention's functions relates to a method for decreasing undesirable bacteria in the intestinal tract for achieving a healthy glucose and insulin regulation, inclusive intestinal and body health, and metabolic fitness. The invention has both human and animal application.

### TECHNICAL FIELD

Health and Beauty

### BEST MODE

Particularly useful, exemplary, and not limiting embodiment of the invention. These four conditions had to be solved when developing and constructing the present invention.

First, the Invention had to be small as to not over expose the surrounding area beyond the external anal sphincter, for over exposing nullifies the desired effect.

Second, the invention had to conform to the contours of the exposed anus. This is an important reason why the invention is one-thirds full with a thick gel with a low water count—for example, a device such as an ice pack with a high water count filled to capacity would not contour enough to create the desired effect. Furthermore, the hardness of an ice pack could cause skin abrasions or injuries.

Third, the invention had to remain at the desired temperature for its size to transmit a penetrating chill of 0° Celsius or lower. Ice and even normal gel packs with a high water count freeze solid at low temperatures, where a gel with a low water count does not transform into a solid state.

Fourth, the invention had to be simple enough so an individual could apply without difficulties during the application process.

Temperature and time duration: The invention's temperature and time duration was derived from the first few discovery applications. Furthermore, temperature and time duration of the invention was confirmed by researching different temperatures: warm, cool and several stages of increasing coldness. The research data supported the facts, only freezing or near freezing temperatures produced the desired result. In addition, using the invention less than 7 seconds was not enough time to permit a penetrating chill to take place needed to cause a reaction. Times longer than 15 seconds did not seem to show any signs of the initial reaction continuing, likely the result of the body taking intestinal counter actions to regain homeostasis.

In the three-week testing period, the Cryo-Gastrointestinal Activator's current construction was used. This change resulted in greater application ease, availability, and effectiveness compared to the freezing water application. The problem with the water application is that water tends to expose beyond the desired area. Another problem to the water application is that it is difficult to have water readily available for this type of procedure at the desired temperature when needed.

Freezing of the invention: Simply place the invention in a freezer be it a home, store, business, or some type of travel freezer, and when needed retrieve the invention from the freezer. Knead the invention filled with the thick gelatinous substance with a low water count to make it pliable. Coat the invention with a contact gel, and then apply it to the area described for 7 to 15 seconds. Discard the invention after use, for the invention was designed as a disposable apparatus.

Construction of the invention: I constructed a 2″ Long, 1½″ Wide, and ½″ High container from a 2-millimeter thick sheet of plastic. The plastic's composition is of a durable cold and heat resistant design, for when the cold treatment is changed to a heat treatment. The container was constructed by using a plastic sealing machine that uses a method for sealing whereby heat is applied to seal. The container is filled one-thirds full with the thick gelatinous substance with a low water count and then sealed.

Use of a thick gelatinous substance: The choice to use a thick gelatinous substance with low water content is because it would congeal but not freeze solid—as an example, ice packs freeze solid, which prevents conforming to body contours and tends to cause skin abrasions and/or injuries. Chemical self-chilling ice packs are too big and bulky that over exposed and never reached the desired temperature. Cold sprays (CO) chills the skin, but does not penetrate enough to reach the hindgut-proctodeum junction without greatly over exposing the surrounding area that results in non-activation of the desired response.

Construction of the handle: The invention has a 2″ Long, 1½″ Wide plastic handle attached used for grabbing and holding the invention between the fingers and thumb. The handle also helps to direct the device into place, and helps in applying directional pressure. Construction of the grabbing and holding handle is accomplished by trimming off the excess plastic from the container, but leaving a 2″ Long, 1½″ Wide trimming on one of the longer sides of the invention that could be used by either right or left-handed people. Although the size and shape are an approximation, they are not to imply limitation.

### HISTORY OF THE INVENTION

Understandably, it is important to learn how the invention came to be. The discomfort caused by repeated IBS episodes prompted the action of using freezing water, being all we had to clean with and help ease the pain. Therefore, an application of freezing water that was near 0° C. was applied to the external anal sphincter. A time of 7 seconds lapsed when I decided to stop, because I could feel the cold temperature penetrating. Afterwards, I went and lied down and waited for the next diarrhea episode. However, instead of another episode, I found myself waking up the next morning feeling refreshed from a good night's sleep. This is a change not to be expected when you are accustomed to having more IBS episodes before it stops.

Discovery of the invention: Upon waking, I instantly knew something unexpected had taken place from of the freezing water application. I decided, the next time I had an IBS episode, I would repeat the process

Pretesting of the invention: A few days later, I had another IBS episode where I repeated the freezing water application. About 5 minutes later, the IBS symptoms stopped. I used the freezing water application a few more times to make sure it stopped the IBS symptoms. Feeling to a certain degree that the cold water has an effect on IBS symptoms, I move to the next logical step.

Testing of the invention: I decided to conduct research and testing of the invention and then document the results daily.

Testing of the invention to see if it could stop an IBS episode from starting: I made a change to the testing procedure; I decided to conduct the freezing water application before the IBS episode started, when I felt an IBS event was about to start. Normally, it was intestinal discomfort that was the indication of an oncoming IBS event. However, later I started to notice that food and liquids tasted strange 12 to 24 hours before an IBS event.

Construction of the present invention for the freezing water application had inherent drawbacks: I started to design a device that could be more practical, easier to use, and available when needed. The result of this design work is the present invention. Although, I have other designs suited for the medical profession, it was however out of my financial ability to build.

A decision was made to dedicate three weeks to research, testing, and documentation of the daily results. Testing was only at first focused on the effect the invention had on diarrhea episodes. Therefore, I conducted a treatment every time I felt an IBS episode was about to start, which stopped all IBS episodes from starting. From the research results, it became clear that the large intestine extracted liquids very quickly when the invention was applied that prevented diarrhea by returning the liquids to the body. I now refer to this as the Reverse Liquid Osmosis Action (RLOA).

A testing discovery: I discovered through an accidental over exposure of the freezing water application beyond the external anal sphincter to the buttocks region that over exposure to the surrounding area cancels the freezing water application's desired results. This is likely because the nerve fibers contained in the skin of the anus and anal canal are not the same as the nerves contained in the greater portion of the body's skin. This information helped in the design of the device for the construction size and applicability was an important factor.

### History of the Inventions Research

The present invention comprises the discovery and development of an effective cryogenic gastrointestinal triggering device that activates the gastrointestinal tract and body functions during the application process. The invention's discovery was the result of positive health changes to symptoms of a health condition known as Irritable Bowel Syndrome or IBS. However, it should be noted that the positive health changes are not limited to Irritable Bowel Syndrome, and have both human and animal application. The invention is made from cold and heat resistant high strength plastic. The reason for using cold and heat resistant high strength plastic is that when the cold treatment is changed to a heat treatment to the same body location there seems to be a change to intestinal activity that initiates a hunger response. The theory is, keeping the body warm constantly mainly the buttocks region and specifically the external anal sphincter keeps the eating response active that in time could lead to obesity and intestinal problems such as reoccurring diarrhea, colic, or other intestinal conditions. In addition, there seems to be a connection between house pets and stabled animals such as horses that are kept warm but have recurring cases of diarrhea. However, it was the discovery resulting from the heat treatment research that revealed something that might help people with Cystic Fibrosis. 12 to 24 hours after having a heat treatment the lungs started expelling mucus that seemed to improve breathing. Understanding the possibilities that could come from the C-G, A afforded the consideration to why people exposed to critical temperatures do not have the same internal changes that come from the Cryo-Gastrointestinal Activator. It became clear to why peoples body's suddenly exposed to low temperatures such as with air (artic wind), water (freezing water), or nitrogen vapors (WHC Whole Body Cryotherapy) do not have the same reaction as to the Cryo-Gastrointestinal Activator. It is because, regardless if peoples actions for exposure are for fun or self-preservation the fight or flight response is activated and adrenaline is released into the body that changes intestinal activity. Furthermore, the threat is external and not internal that changes how the body reacts.

The unique characteristic of the invention is that it is not ingested, injected, or inserted into the body. The present invention is applied to a specific part of the body the external anal sphincter be it human or animal. The invention's benefits are positive health changes during and following the application process from increased intestinal activity, or hyper-digestion.

Treatment for Irritable Bowel Syndrome: The invention's discovery was the result of noticeable changes in active Irritable Bowel Syndrome symptoms. The changes to IBS symptoms were noticed at the time the invention was applied, although the final construction stage of the invention was still in development. In addition to IBS changes, there were changes to other related and seemingly unrelated health conditions following the application of the invention during the days and months afterwards.

Stages of Discovery

The changes that were noticed took place prior in the discovery stage of the invention: When noticing a change took place but before recognizing and understanding the value of the Cryogenic Gastrointestinal Activator triggering device.

Prior to research in the investigation stage of the invention: The invention's values being recognized during this stage of testing of the Cryogenic Gastrointestinal Activator, triggering device.

Planned research and testing stage of the invention: Research and testing the values of the Cryogenic Gastrointestinal Activator, triggering device. And

Post research and testing of the invention: Understanding the need for continued research and testing based on furthered discovered values of the Cryogenic Gastrointestinal Activator, triggering device.

Personal history: I suffered from a condition now known as Irritable Bowel Syndrome since I was a young teenager until Jan. 21 2016, which coincides with the time the research and testing of the invention ended. The prevalent symptoms of Irritable Bowel Syndrome were diarrhea and abdominal pains, which the research focused on.

Unfortunately, in the initial research simple answers were not forthcoming, which prompted a more in-depth study of the intestinal tract to what caused the Irritable Bowel Syndrome symptoms [1] to quickly reduce during active IBS episodes. I felt an answer could be obtained quickly because unlike researchers in an attempt to find a cure, I believed I had a cure for this medical condition. So now, firm in my belief, I started researching backwards from cure or more correctly cures to what caused these positive changes to occur, since numerous health conditions showed signs of improvement and or ceased to continue.

Two sections in a chapter from an Anatomy and Physiology textbook provided the foundation or basic understanding to the cause and effect of the body changes [2] [26]. Understanding was then advanced by other research articles.

First basic understanding: This reference [2] explains a natural involuntary intestinal action, or reflex that I discovered can be artificially created. For information purposes, involuntary intestinal action or reflex is located in the large intestine near the ileocecal sphincter. The ileocecal sphincter is at the juncture between the ileum and the large intestine and remains somewhat contracted most of the time. However, peristaltic waves reaching the ileocecal sphincter from the small intestine cause the sphincter to lessen the contraction allowing chyme to move from the small intestine into the cecum, which is the proximal end of the large intestine.

Cecal distention: Cecal distention is an action of stretching out the cecum as it fills with chyme, which initiates a local reflex that causes more intense constriction of the ileocecal sphincter causing its closure. The intestinal action of filling the cecum facilitates digestion and absorption of chyme in the small intestine and slows the rate of chyme movement from the small intestine into the large intestine by preventing intestinal content from returning to the ileum or small intestine.

When artificial cecal distention is created, the brain receives a signal through neural pathways that is perceived as a stretching out of the cecum, which causes the sphincters to close. This is initiated by artificially creating intestinal nerves to fire when the Cryo-Gastrointestinal Activator device is applied to the external anal sphincter during the application process. Thus, the body reacts especially in the intestinal tract by the penetrating chill the device creates. The invention's severe penetrating chill anal contact triggers pain [3] and thermoreceptors [4] on the external anal sphincter and within the anal canal where skin and intestinal tract meets, which includes the internal anal sphincter. The cold affected pain and thermoreceptors initiate a response through neural pathways that registers in the brain as a severe core temperature change or threat during the application process.

Body core temperature threat: Within the anal canal, between the external and internal anal sphincters where the intestinal tract ends and skin begins is the only place on the body where a penetrating chill could cause an intestinal core temperature threat externally making this body location with the accompanying invention unique.

Neutralization of bacterium interference: As a core temperature change is detected, the body reacts to contain heat and reverse the apparent threat by making changes in several areas of the body. Application of the invention creates firing of the nerves artificially within the intestinal tract that produces a sensation that the mind interprets as a local reflex in the cecum that closes the ileocecal sphincter. The artificially created firing of the nerves [5] causes the small intestine to facilitate a more intense form of digestion, absorption, and transport of intestinal content that also slows the rate of chyme movement. The result of the artificially created firing of the nerves causes hyper-digestion that improves an improperly functioning intestinal tract by neutralization and removal of bacterium interference.

The intense activation of the intestinal tract last only for a short time. However, the activation is long enough to neutralize bacteria with greater amounts of digestive fluids before the action of intestinal digestion is slowed to prevent digestive sweating. Digestive sweating is a natural action of cooling the body, but is counterproductive to heat retention needed to counter the core temperature threat. The body also counters digestive sweating by restricting or closing sweat glands, which also helps with or cures Hyperhidrosis [6] as in my case. In addition, besides curing Hyperhidrosis there were other changes seen and felt. The body wide reaction to the invention's intestinal cold application seemed to cause the body's entire nervous system with improved intestinal functions to stimulate finger and toe nails and hair to grow thicker and shinier, some pigmentation return to the hair, a reversal to a receding hair line, muscle growth, a reduction in chronic back pain from a back injury, and faster healing of injuries. It is my theory that the reaction to the invention, especially the nerve reaction could be of benefit to people with MS-Multiple Sclerosis, and maybe with Epilepsy.

The manual action of artificially activating the intestinal tract results in lower amounts of bacterial in the small intestine by hyper-digestion that also impedes further bacterial growth. The invention also removes bacteria by mucus coating or encasement, a natural intestinal process. Mucus production increases with artificially created hyper-digestion resulting in improved intestinal functions. Furthermore, it should be noted that artificial cecal distention could be created with the invention's application without having diarrhea or IBS symptoms in order to help with other health conditions like Birth Defects caused by intestinal bacterial.

When the invention is applied, the intestinal tract produces greater amounts of digestive fluids, and mucus that protects the intestinal wall, where the lack of mucus production is believed to be the cause of intestinal discomfort. In a second stage, the small intestine counters greater digestive fluids by reducing the pH levels—as an example, when the pH of the stomach contents falls below 2, increased gastric secretion produced by distention of the stomach is blocked. This negative-feedback mechanism limits the secretion of gastric juice. In the case of the small intestine, the result is an improved functioning intestine by the removal of bacterial, which result in restoring diminished digestion, absorption, and transport of intestinal content. The outcome of the invention's action is over all body health, especially to the immune system (immunotherapy) to fight cancer [7].

The invention works by biofeedback (nerve systems). There are two types of nerve networks working in the intestinal tract—extrinsic nerves and intrinsic nerves [8]. Extrinsic nerves come from outside the digestive system. They connect the digestive organs to the brain and the spinal cord passing information back and forth. This is how the artificial firing of the intestinal nerves registers in the brain.

Two main chemicals are released into the digestive system by these nerves, acetylcholine, and adrenaline. Acetylcholine causes the muscle layer of the digestive organs to squeeze creating the action called peristalsis, which pushes the food and juices through the digestive tract. Acetylcholine also causes the stomach and pancreas to produce greater quantity of digestive juices.

The intrinsic nerves are a dense network of neurons, called the enteric nervous system, which are embedded in the walls of the esophagus, stomach, small intestine, and colon. The nerves are triggered to act when the walls of these organs are stretched by food. Research has revealed that the Cryo-Gastrointestinal Activator device can also trigger these organs artificially during the application process. The invention's artificial triggering of the intestinal tract facilitates the release of many different substances (Chemicals) [9] [10] that can speed up or delay the movement of food and the production of juices by digestive organs. In addition to the release of many different chemicals, there seems to be greater production of chemicals that for some reason reduced or stopped. When these chemicals are reactivated they led to the improvement of certain health conditions like, PDS Post Dramatic Stress syndrome, chronic stress and fatigue, high blood pressure, and a reduction to the over sensitized or chronic adrenaline dosing. There was improvement to Lactose Intolerance a conditions caused by the lack of the enzyme lactase that could only come from the improved production of this enzyme. Then there was the return of a natural intolerance to pain medications and alcohol that led to consumption reduction and then to non-dependency of these addictive substances. Furthermore, there was a reduced desire for over consumption of sweets and salts. A dramatic diminishing of a condition known as agoraphobia, and an improvement to a reading and writing condition known as dyslexia as well as other mental functions. There were signs of improvement to a mental condition known as bipolar, and to a condition known as acne. My theory is, with the improvement to the intestinal tract the Blood Brain Barrier has an increased selection of molecules such as glucose and amino acids that are crucial to neural functions.

The intestinal neural network is in constant communication with the rest of the body. The system works within close partnership with the immune system, endocrine system, and circulatory system and contains over a billion neurons, more than in either the spinal cord or the peripheral nervous system. This multitude of neurons in the enteric nervous system enables us to detect the workings of our intestinal tract and its contents. The enteric nervous system uses more than 30 neurotransmitters, just like the brain.

The body's nerve system is so vast and complex that it can detect the slightest temperature change especially in the intestinal tract. This is the reason why the Cryo-Gastrointestinal Activator device's penetrating cold contact can cause a detectable core temperature change or threat during the application process. The body responds to the temperature change when detected with quick countering actions like sending more blood to warm the cold affected body parts, closing sweat glands, and intestinal sphincters. The response also releases chemicals in the brain that calms the body and initiates a strong satisfied hunger state to prevent eating or drinking, and also prevents the releases of certain chemicals such as adrenaline.

Adrenaline has the opposite effect as Acetylcholine. It relaxes the digestive muscles and decreases the flow of blood to these organs, which slows or stops digestion. Because adrenaline activates the flight or fight response it causes all normal processing of the body to slow down or stop, so that in case of an emergency blood could be diverted to the extremities for running or fighting.

Digestive problems: Stress is one of the things that activate the adrenaline response. If stress continues over long periods, digestive problems are likely to result. Irritable Bowel Syndrome is one of those problems, because the digestive system slows or shut down all the time stress is experienced, which permits intestinal bacteria to grow during slowed or in times of inactivity.

Conducting an artificial triggering of the intestinal tract: The result of the penetrating chill causes a core temperature change that is detected as an actionable internal emergency causing body functions to initiate a digestive response in the process, however not an adrenaline response. The triggering causes the body to respond in a manner that compartmentalizes the intestinal tract in order to retain heat by closing the intestinal gates (sphincters)—as an example, the body responds to the invention's application by closing the lower esophagus sphincter as well to conserve heat in an attempt to prevent food or liquid consumption. However, what this action did was to prevent stomach content from entering the esophagus, which prevents heartburn from occurring. This action was also the first change of a health condition noticed, discontinued acid reflux.

Research of the body's anatomy helped in understanding how the intestinal nerve system communicates with the rest of the body. However, it did not explain how placing a severely cold device on the external anal sphincter created a trigger.

Answer—the external anal sphincter is the closes point on the body to the intestinal tract [11] [12]. Just within the anus is the pectinate line [13] and Intersphincteric groove [14]. The pectinate line divides the upper two thirds from the lower one third of the anal canal. This line represents the hindgut-proctodeum junction, a point where the mucosa of the intestinal tract becomes skin [15] [16] [17] [18] [19] [20].

In order to understand the triggering of the intestinal tract, there was a need for more understanding of the body's anatomy. The term gastrointestinal tract technically only refers to the stomach and intestines, but is often used as a synonym for the digestive tract. The digestive tract is a tube extending from the mouth to the anus, and its associated accessory organs, primarily glands, which secrete fluids into the digestive tract [21].

The digestive tube consists of four major layers, or tunics: (a) an internal mucosa and (b) an external serosa with (c) a submucosa and (d) muscularis in between. These four tunics are present in all areas of the digestive tract from the esophagus to the anus. Three major types of glands are associated with the intestinal tract: (1) unicellular mucous glands in the mucosa, (2) multicellular glands in the mucosa and submucosa, and (3) multicellular glands (accessory glands) outside the digestive tract.

The innermost tunic, the mucosa, consists of three layers: (1) the inner mucous epithelium, which is moist stratified squamous epithelium in the mouth, oropharynx, esophagus, and anal canal and simple columnar epithelium in the remainder of the digestive tract; (2) a loose connective tissue called the lamina propria; and (3) an outer thin smooth muscle layer, the muscularis mucosae.

The submucosa is a thick connective tissue layer containing nerves, blood vessels, and small glands that lies beneath the mucosa. The plexus of nerve cells in the submucosa form the submucosal plexus, a parasympathetic ganglionic plexus consisting of axons and many scattered cell bodies.

Treatment for intestinal bacterial: The artificial triggering of the intestinal tract by the invention initiates a myriad of involuntary body actions, countering actions, and reactions. As an example, repeating the triggering of the intestinal tract causes digestive juices including insulin to flow and digestive actions to initiate and not just, where the intestinal tract detects the presents of intestinal content, but throughout the entire tract. As a result, in the small intestine, bacterial [22] is digested and naturalized causing the bacterial to lose its hold in a type of intestinal flushing, whereby reducing bacterial over growth [23].

Type I Diabetes Mellitus: In patients with type I diabetes mellitus, insulin is lacking resulting in insufficient glucose being transported into the cells of the body. As a result, the cells do not have enough energy for normal function; blood glucose levels become significantly elevated, causing abnormal amounts of glucose to be released into the urine.

Treatment for diabetes by improved insulin production: [24] [25] Although, the invention relates to a treatment for IBS by producing intestinal fluids needed to reduce bacteria, it also produces insulin in conjunction with other digestive fluids that helps with diabetes. The mention of diabetes helps in understanding the overall achieving of a healthy glucose and insulin regulation, and immune system activity, inclusive intestinal and body health, and metabolic fitness.

Diffusion: Webster's New Encyclopedic Dictionary states, diffusion as the intermingling of the particles of liquids, gases, or solids because of their spontaneous movement, so that in dissolved substances they move from a region of higher to one of lower concentration. Example of diffusion, monosaccharides such as glucose and galactose are taken up into intestinal epithelial cells by cotransport, powered by a sodium ion gradient. Monosaccharides such as fructose are taken up by facilitated diffusion. The monosaccharides are transferred by facilitated diffusion to the capillaries of the intestinal villi and are carried by the hepatic portal system to the liver, where the nonglucose sugars are converted to glucose. Glucose enters the cells through facilitated diffusion. The rate of glucose transport into most types of cells is greatly influenced by insulin.

Second basic understanding: Article [26] explains the body function known as diarrhea as a condition of the large intestine being irritated and inflamed. An abnormally frequent discharge of fluid feces is called diarrhea. This is where intestinal mucosa secretes large amounts of mucus, electrolytes, and water by movement called osmosis into the colon.

Treatment for diarrhea: When the invention is applied during, an active diarrhea episode or prior to, the application reverses water movement by osmosis into the colon. The normal action of liquid absorption by osmosis from the colon into the body recommences, hence slowing, stopping, or preventing an active diarrhea episode. The research conducted helped in understanding the Reverse Liquid Osmosis Action (RLOA).

In conclusion, although, research focused on the effect the invention had on Irritable Bowel Syndrome symptoms, it is now believe the invention has greater potentials that is particularly useful, and not limiting. Example, the artificial triggering also initiates a strong satisfied hunger feeling from leptin, the satiety hormone during the application process to prevent eating, an action that aids with heat retention. This action also restores correct eating functions, a correction that helps in preventing over eating, also known as an eating disorder.

### RELATED ART

Failing to find any information that relates to the invention, the related art focused on its effect on health conditions. Health—it is an unfortunate and all too common occurrence that when a person has a gastrointestinal problem like IBS they are limited in options, options that normally state, for temporary relief only. These temporary relief options, are over the counter or doctor prescribed pills, liquids, and suppositories to aid in suppressing intestinal symptoms, options like antacids, acid reducers, anti-diarrhea, stool softeners, laxatives, or any number of natural health food (mixtures) trying to help with acid reflux, indigestion, intestinal pains, cramping, or diarrhea.

Then there are the research labs around the world trying for years to cure the intestinal health condition, Irritable Bowel Syndrome with no results. Although every now and then there is a break through, something is discovered that is said to help one or two of these problems, but only temporarily that join the list of things that might help intestinal problems.

A similar problem exists with experimental surgeries meant to help with these intestinal health conditions—for example, removal of part of the small intestine or the colon that is sure to condemn people to the very medicines they were trying to be relieved from; or worse, a feeding tube or colostomy bag.

The options given has taught a variety of substances are available for intestinal needs. Examples of intestinal substances available include, have at home or carry-a-long antacids for when unexpected acid reflux is encountered. Other substances include acid reducers meant to be taken before meals but often causes diarrhea. Anti-diarrhea medicines for when the intestines are in a state of uncontrolled discharge. Stool softeners or laxatives to restart a stalled intestinal tract, which is normally the result of diarrhea medicines. Many of these substances are only marginally effective, because they depend upon the user to control. The problem with self-administering these substances is that they are taken after an intestinal condition starts, and not before to stop the intestinal problem from occurring at all, as in the case of the invention.

An additional problem is that people in intestinal discomfort expect quick relief, and often overuse to shorten the distress even when these medicines normally state the substance needs time to enter the body before they work. The invention causes the gastrointestinal sphincters to react at the time of the application, where people felt relief within seconds and total recovery in most cases from the symptoms within minutes.

Example of an effective treatment for intestinal symptoms as stated above would be to continue the invention's applications until the gastrointestinal sphincters have regained their closing strength, which is similar to performing physical exercise. A problem with the invention is that it is only effective in preventing intestinal symptoms, if used. Intermittent use is only useful to people with occasional symptoms such as heartburn, indigestion, or diarrhea brought on by bad food choices, stress, or medicines.

These options also teaches a permanent dependency on temporary relief medicines. Unfortunately, temporary relief options are only effective some of the time. Furthermore, temporary relief options do not claim long-term improvement to intestinal health conditions.

Accordingly, there exists a need for a method and apparatus for combatting the common range of intestinal symptoms that does not require ingesting or inserting substances, or the need for surgery. Furthermore, there is a need for such a method and apparatus for many people, who are allergic to certain chemicals or chemical combinations.

### SUMMARY OF THE INVENTION

The invention comprises a method and apparatus for activating the gastrointestinal tract. The invention utilizes cryogenics (Etymology, The word cryogenics stems from Greek (cryo)—“cold”+(genic)—“having to do with production”.), a term used to denote the production and effects of low temperatures, although, standard cryogenic temperatures are much lower. The invention is applied to the exterior anal sphincter. The invention is made from cold and heat resistant high strength plastic. When the invention is applied, it triggers the nerves to start firing. Signals are sent from the triggered nerves via the gastrointestinal tract nerve system and other neural pathways to the brain, which responds by causing sphincters to contract or close. The gastrointestinal tract starts a state of hyper-digestion, causing it to produce digestive juices also that digest and neutralizes intestinal bacterial. Hyper-digestion is the production of digestive fluids that is not dependent only on intestinal content activation.

### BRIEF DESCRIPTION OF THE DRAWINGS

These and other features and advantages of the present invention will become more readily appreciated as the same becomes better understood by reference to the following detailed description when considered in connection with the accompanying drawings, wherein:

FIG. 1 is a top elevation view showing FIG. 1A the body of the subject invention, a deforming sealed container filled one-thirds full with a gel with a low water count that does not freeze solid at freezing temperatures that permits it to conform to the indicated location at the desired temperature. FIG. 1B is the handle of the subject invention, used for gripping, holding, and directional pressure.

FIG. 2 is a side view showing FIG. 2A the body of the subject invention, and FIG. 2B the handle of the invention.

### DETAILED DESCRIPTION OF PREFERRED EMBODIMENT

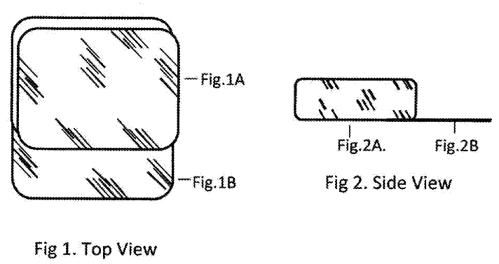

The purpose of the two figures of the invention, FIG. 1 the top view, and FIG. 2 the side view, which of both show, FIG. 1A and FIG. 2A the body of the invention, and FIG. 1B and FIG. 2B the handle of the invention is of illustration and not to be in any way limiting. Referring to FIGS. 1 and 2: FIG. 1B and FIG. 2B the handle, it will be appreciated that the invention can be equally applied with the left or right hand.

Referring to FIG. 1: FIG. 1 is a top elevation view. This view shows FIG. 1A the body, a sealed container capable of deforming. The container is one-thirds full with a thick gel with a low water count so it will not freeze solid at temperatures at or below 0° C., which permits it to stay in its liquefied state so it can conform to the indicated location at the desired temperature. In addition, FIG. 1B the handle of the invention is made from the same material, all of which is one piece of plastic. FIG. 2 is a side view showing FIG. 2A the body, and FIG. 2B the handle of the invention for gripping, holding, and directional pressure.

The invention has been described in an illustrative manner, and it is to be understood that the terminology, which has been used, is intended to be the nature of words of description rather than of limitation. Obviously, many modifications and variations of the present invention are possible in light of the above teachings. For example, the subject invention FIG. 1B can be adapted to be applied with the left or right hand and is not limited shown in the illustrations. It is therefore, to be understood that within the scope of the appended claims, the invention may be practiced otherwise than as specifically described. The invention is defined by the claims.

What is claimed is:

1. A device that activates more the gastrointestinal tract into hyper-digestion by means of cryogenic gastrointestinal triggering of involuntary intestinal and non-intestinal body functions during the application process; a device that activates more the gastrointestinal tract into hyper-digestion means the production of digestive fluids above normal than would be produced under normal activity; a device that activates the gastrointestinal tract involuntary intestinal and non-intestinal body functions means involuntary intestinal and non-intestinal body functions that cannot be controlled by self-will; a device that creates artificial actions or reactions; a device that creates artificial actions or reactions means actions or reactions produced other than normal involuntary processes; a device that triggers the activation of the gastrointestinal tract by means of a penetrating cold temperature resulting from the chilled content of the flexible sealed container that creates the intestinal nerves to fire by artificial means; a device that triggers nerves into firing a signal by means of a penetrating cold temperature produced during the application process; a device a flexible sealed container that is applied to a specific part of the body; a device that is applied to a specific part of the body means the external anal sphincter, a device that transmits a cold temperature externally to target an intestinal section the hindgut-proctodeum junction during the application process; hindgut-proctodeum junction means where the intestinal tract ends and the skin begins just inside the anal canal; a device that transmits a cold temperature to the body that results in internal actions and reactions, comprising: a flexible sealed container that contains a thick gelatinous substance with a low water count; a flexible sealed container that contains a thick gelatinous substance with a low water count means a thick gelatinous substance with a low water count that congeals but does not transform into a solid state at freezing temperatures; a thick gelatinous substance with a low water count contained within a flexible sealed container that congeals means the content of the flexible sealed container does not harden at freezing temperatures that permits the content and flexible sealed container to conform to the contours of the external anal sphincter; a flexible sealed container that is filled one-thirds full with a thick gelatinous substance that permits conforming to body contours; a flexible sealed container having a top and bottom and four sides; a flexible sealed container having four sides two of which extends longer than the other two; a flexible sealed container forming an enclosure; a flexible sealed container having top and bottom and four sides' means protectively enclosing the content a thick gelatinous substance with a low water count from the surrounding environment; a flexible sealed container having a top and bottom and four sides each being defined by deforming material able to readily yield to the weight of a person's application thereon; a sealed container with an extension of the deforming material on one of the two longer sides means material extending from a side of the flexible sealed container; and a sealed container with a protrusion of deforming material that forms a handle for gripping, holding, and applying directional pressure in the application process.
